(19) United States
(12) Patent Application Publication
(10) Pub. No.: US 2024/0382344 A1
(43) Pub. Date: Nov. 21, 2024

(54) OPTIONAL SUPPORT FOR OPHTHALMIC DEVICE

(71) Applicant: Edwin Ryan, St. Paul, MN (US)

(72) Inventor: Edwin Ryan, St. Paul, MN (US)

(21) Appl. No.: 18/666,448

(22) Filed: May 16, 2024

Related U.S. Application Data
(60) Provisional application No. 63/466,975, filed on May 16, 2023.

(51) Int. Cl. A61F 9/007

(52) U.S. Cl. CPC A61F 9/007

(57) ABSTRACT

A support device and associated methods for an ophthalmologic instrument are shown. Examples include interior profile elements configured to substantially match an exterior profile of a distal end of an ophthalmologic instrument. Examples also include a latching mechanism to hold a support device to a distal end of an ophthalmologic instrument. Examples also include an interior surface formed from a resilient material.

### CLAIM OF PRIORITY

This patent application claims the benefit of priority, under 35 U.S.C. § 119(e), to U.S. Provisional Patent Application Ser. No. 63/466,975, entitled “Optional Support for Ophthalmic Device,” filed on May 16, 2023, which is hereby incorporated by reference herein in its entirety.

### TECHNICAL FIELD

This invention relates to small gauge instruments typically used for surgical procedures such as surgery of the eye.

### BACKGROUND

Ophthalmological surgery continues to evolve towards smaller instruments that produce smaller incisions. A common incision size currently is 25 gauge. Other instruments as small as 27 gauge (approximately 0.41 mm diameter) are being utilized, and smaller instruments are likely in the future. The advantages of smaller incisions are multiple, including lessened trauma, faster healing, faster wound management (no sutures), and greater patient comfort.

Problems exist with the smaller instruments, however. The small diameter of the instruments makes them quite flexible, which is a disadvantage for the surgeon. With larger diameter instruments, there is very little “play,” so the tips of the instruments go exactly where the surgeon desires that they go. With the smaller diameter instruments, the tips can move from their intended positions due to the bending or flexing of the fine wire-like instruments, which makes the surgeon feel a loss of control.

When the eye is turned to extremes to allow bending or flexing of the small instruments is of particular concern in some procedures, for example, removal of peripheral vitreous, when the eye must be turned to allow viewing by the surgeon. Turning of the eye is accomplished by moving the instrument relative to the patient's head while a portion of the instrument remains inserted within a portion of the eye. Because the amount of flexing of the instrument is relatively large and unpredictable to the surgeon, precise repositioning of the eye becomes more difficult. In addition, delicate maneuvers such as peeling membranes from the retinal surface become significantly more difficult when instruments are too flexible causing imprecision of movements.

What is needed is an instrument design that accommodates increasingly small diameters, and still provides precise control without unwanted flexing.

### BRIEF DESCRIPTION OF THE DRAWINGS

FIG. 1 shows an example ophthalmological instrument and a separate, removable support device according to an embodiment of the invention.

FIG. 2A shows an example of a support device according to an embodiment of the invention.

FIG. 2B shows an isometric view of another angle of the support device from FIG. 2A according to an embodiment of the invention.

FIG. 2C shows a cross section view of the support device from FIG. 2A according to an embodiment of the invention.

FIG. 2D shows a top view of the support device from FIG. 2A according to an embodiment of the invention.

FIG. 3A shows a support device and a forceps in a stage of connection according to an embodiment of the invention.

FIG. 3B shows the support device and the forceps from FIG. 3A in another stage of connection according to an embodiment of the invention.

FIG. 3C shows the support device and the forceps from FIG. 3A in another stage of connection according to an embodiment of the invention.

FIG. 3D shows the support device and the forceps from FIG. 3A in another stage of connection according to an embodiment of the invention.

FIG. 4 shows an example method of connecting a support device to an ophthalmological instrument according to an embodiment of the invention.

### DETAILED DESCRIPTION

In the following detailed description, reference is made to the accompanying drawings which form a part hereof, and in which is shown, by way of illustration, specific embodiments in which the invention may be practiced. In the drawings, like numerals describe substantially similar components throughout the several views. These embodiments are described in sufficient detail to enable those skilled in the art to practice the invention. Other embodiments may be utilized and structural, or logical changes, etc. may be made without departing from the scope of the present invention.

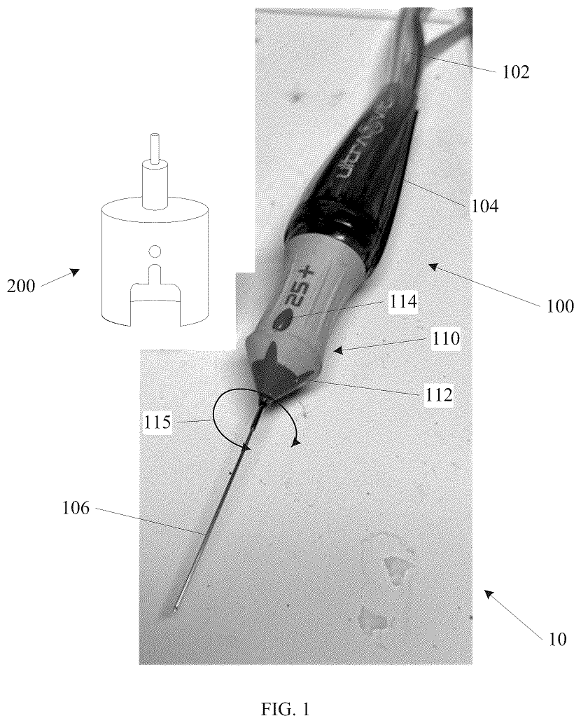

FIG. 1 shows an ophthalmologic system 10. The system 10 includes an ophthalmologic instrument 100 and a removable support device 200. The ophthalmologic instrument 100 includes a base unit 104. Selected examples of ophthalmologic instrument 100 include supply lines 102, such as, fiber optics, infusion, suction, etc.

The ophthalmologic instrument 100 includes a distal end 110 and a small diameter instrument 106 that extends from the distal end 110. The small diameter instrument 106 can be chosen from any of a number of ophthalmologic devices. Examples include, but are not limited to, fiber optic probes, laser guides, suction/cutting tools, forceps, scissors, etc. In one example, the small diameter instrument 106 includes a hollow tube. In one example, the small diameter instrument 106 has a diameter smaller than 20 gauge. In one example, the small diameter instrument 106 has a diameter equal to, or smaller than 23 gauge. In one example, the small diameter instrument 106 has a diameter of approximately 25 gauge. In one example, the small diameter instrument 106 has a diameter of approximately 27 gauge.

In the example shown, the distal end 110 includes a cone shaped portion 112. The cone shaped portion 112 is used for secure, but reversible attachment. The cone shaped portion 112 is useful for a surgeon to better hold and control an orientation and position of the small diameter instrument 106. In the example of FIG. 1, the distal end 110 further includes a protrusion 114. The protrusion 114 is useful for a surgeon to feel, and know a rotational orientation indicated by arrows 115. The protrusion 114 also provides additional grip when rotating the ophthalmologic instrument 100.

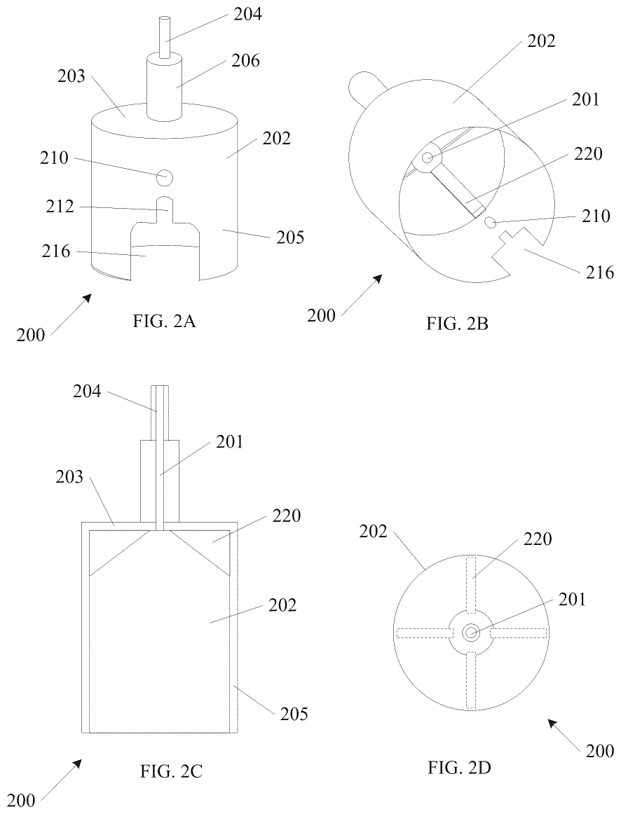

FIG. 2A-2D show additional detail of the support device 200 shown in the ophthalmologic system 10 of FIG. 1. In FIG. 2A, the support device 200 includes a cylindrical base 202. A support 204 is shown extending from the cylindrical base 202. In one example, the support 204 has an interior dimension that is configured to fit closely around an exterior dimension of a small diameter instrument such as the small diameter instrument 106 from FIG. 1. In one example, the support 204 is a metal tube that is dimensioned to fit closely around the small diameter instrument 106. Although a tube is shown as an example, the invention is not so limited, other structures, such as square cross section, lateral contacting strut of material, etc. are within the scope of the invention. Although metal is used as an example, other possible materials include polymers, composite materials, etc. Stainless steel, or titanium are good metal choices, as they are non-reactive in a human tissue/body fluid environment. In one example, a proximal end of support 204 is cylindrical, so the tips of small diameter instrument 106 can pass smoothly and atraumatically through as the support device is placed on the instrument.

The example cylindrical base 202 includes a solid top portion 203 and a sidewall portion 205. Although a solid cylindrical base 202 is shown, other examples may include additional cutouts in the cylindrical base 202. In one example, the cylindrical base 202 is formed from a continuous integral material. In one example, the cylindrical base 202 is formed from a continuous integral polymer. Polymer materials better facilitate manufacture, in methods such as injection molding. Polymer materials can also be formulated with desired mechanical properties, such as an amount of resilience. A particular level of resilience provides structural support, while also providing a retaining force for latching mechanisms or other retaining mechanisms such as an interference fit as discussed in more detail below.

An intermediate support portion 206 is shown in the example of FIG. 2A. This adds further enhanced stability to the support 204, while still allowing a surgeon good visibility to the small diameter instrument 106. In one example the intermediate support portion 206 and the cylindrical base 202 are integrally molded from a polymer material around a metal support 204. This combination provides improved stiffness from the metal support 204 while also providing a desired resilience from the cylindrical base 202 for retaining or latching the support device 200 to the ophthalmologic instrument 100 as described in more detail below.

In one example, the cylindrical base 202 includes a recess 210. Examples of the recess 210 include holes that pass all the way through the sidewall 205 of the cylindrical base 202, or the recess 210 may only pass partway through the sidewall 205 of the cylindrical base 202. In one example, the recess 210 aligns with the protrusion 114 from the distal end 110 of the ophthalmologic instrument 100. In one method of connecting a support device 200 to an ophthalmologic instrument 100, the support device 200 is aligned with the small diameter instrument 106 extending from the distal end 110 of the ophthalmologic instrument 100. The support 204 extending from the cylindrical base 202 of the support device 200 is slid over the small diameter instrument 106. The recess 210 is aligned with the protrusion 114, and with an amount of engaging force, the recess 210 clicks into engagement with the protrusion 114. Although an example is shown with a protrusion 114 on the ophthalmologic instrument 100, and a recess 210 on the cylindrical base 202, the locations can also be reversed. A protrusion may be located on the cylindrical base 202, and a recess may be located on the ophthalmologic instrument 100. One advantage of having a tactile feature (either a protrusion 114 or a recess 210) that is exposed on an outside of the support device 200 is that a surgeon retains the ability to feel an orientation of the ophthalmologic instrument 100 even with the presence of the support device 200 over the distal end 110 of the ophthalmologic instrument 100.

In one example, the recess 210 interacts with a feature of the ophthalmologic instrument 100 that serves a dual purpose, such as the protrusion 114 as shown in FIG. 1. As discussed above, a protrusion 114 may be included as part of an ophthalmologic instrument 100 for a surgeon to feel, and know a rotational orientation indicated by arrows 115 in FIG. 1. Efficiencies in manufacturing are gained by using an included feature such as the protrusion 114 to also serve as a latching mechanism with the recess 210.

A level of resilience is selected by choosing an appropriate material for the cylindrical base 202. One of ordinary skill in the art, having the benefit of the present disclosure, will recognize that a resilience high enough to provide a desired stiffness to the support 204 and hold the small diameter instrument 106 to at least a level as stiff or stiffer than an equivalent 20 gauge instrument is desired. At the same time, a level of resilience is selected for the cylindrical base 202 to permit deformation over the protrusion 114, and engagement with the recess 210.

In one example, a first cutout 216 is included in the sidewall 205 of the cylindrical base 202. A second cutout 212 may also be included in the sidewall 205 of the cylindrical base 202. The inclusion of one or more cutouts 212, 216 further adjusts an amount of deformation in the sidewall 205 of the cylindrical base 202, which further facilitates connection of the support device 200 to the ophthalmologic instrument 100.

FIG. 2B further shows one or more interior profile elements 220 configured to substantially match an exterior profile of the distal end 110 of the ophthalmologic instrument 100. In the example of FIGS. 2A-2D, four interior profile elements 220 are shown. In one example, the interior profile elements 220 are configured to substantially match at least a portion of the cone shaped portion 112 of the distal end 110. A cone shaped configuration in particular provides easy mechanical alignment of the support device 200 with the distal end 110 of the ophthalmologic instrument 100, and further provides improved mechanical properties, such as resisting any side to side twisting of the support device 200 with respect to the ophthalmologic instrument 100. The angle of sides of the cone provides both lateral support, and a centering function. Thus, by matching the cone shaped portion 112 with the interior profile elements 220, structural support of the small diameter instrument 106 is improved. FIGS. 2C and 2D further illustrate the example geometry of the one or more interior profile elements 220.

Although a cylindrical base 202 is shown, the invention is not so limited. Other example shapes, such as a bullet shape, tapered shape, etc. are within the scope of the invention. Any shape for base 202 can be mounted securely to an ophthalmologic system, such as system 10, using interior profile elements such as interior profile elements 220 as described above.

In one example, the one or more interior profile elements 220 are radially spaced apart at equal angular spacings from one another. For example, with four interior profile elements 220, each interior profile element 220 is approximately 90 degrees apart from other interior profile elements 220. With three interior profile elements 220, each interior profile element 220 is approximately 120 degrees apart from other interior profile elements 220. Equal spacing provides consistent support from all sides of the system 10.

Although a cone shaped profile of the distal end 110 of the ophthalmologic instrument 100 is used as an example, the invention is not so limited. Other profiles of the distal end 110 of the ophthalmologic instrument 100 and corresponding interior profile elements 220 are also within the scope of the invention.

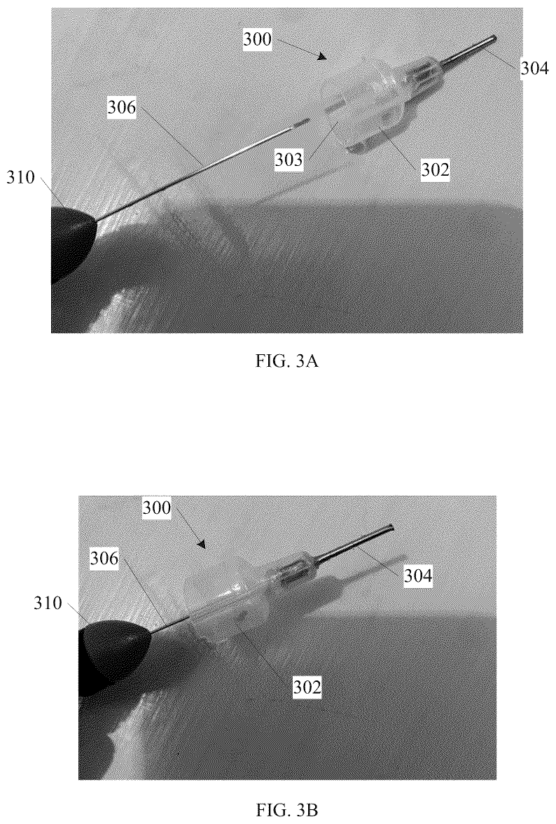

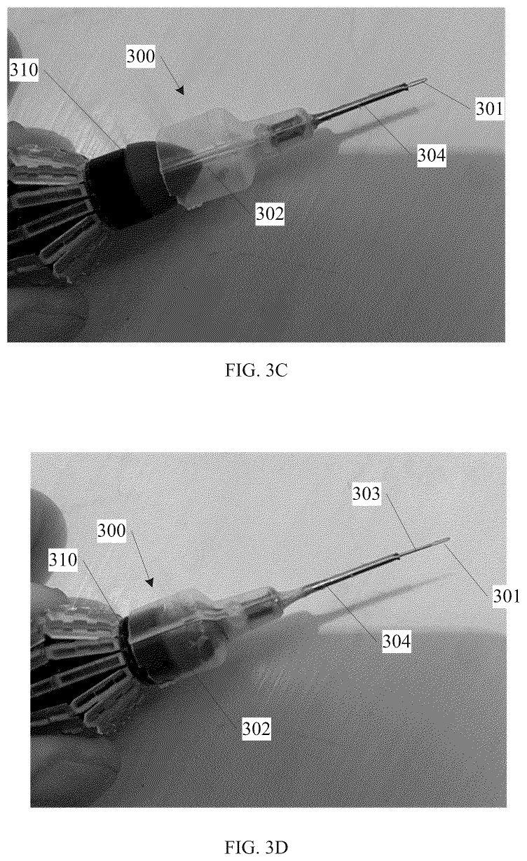

FIGS. 3A-3D shows a number of successive stages of connection for a support device 300 and a distal end 310 of an ophthalmologic instrument. In FIG. 3A, the support device 300 is shown, including a cylindrical base 302 and a support 304. In one example, the cylindrical base 302 includes at least a portion of an interior surface 303 formed from a resilient material. The interior surface includes an interior dimension that is configured to provide an interference fit with the distal end 310 of the ophthalmologic instrument. In one example, the cylindrical base 302 is integrally formed from the resilient material, although the invention is not so limited. In one example, an interior liner of the cylindrical base 302 is formed from the resilient material. In one example only a portion of interior surface 303 includes the resilient material. One advantage of the cylindrical base 302 being integrally formed from the resilient material includes lower cost manufacturing.

In one example, the support 304 includes an interior dimension that is configured to fit closely around an exterior dimension of a small diameter instrument 306 extending from the distal end 310 of the ophthalmologic instrument. In the example of FIGS. 3A-3D, the small diameter instrument 306 includes a forceps, although the invention is not so limited. Other examples of small diameter instruments 306 include, but are not limited to, fiber optic probes, laser guides, suction/cutting tools, scissors, etc. In one example, a proximal end of support 304 is cylindrical, so the tips of small diameter instrument 306 can pass smoothly and atraumatically through as the support device is placed on the instrument.

In FIG. 3B, the support device 300 is aligned with the small diameter instrument 306 extending from the distal end 310. The small diameter instrument 306 is shown beginning to pass through the support 304. In FIG. 3C, the small diameter instrument 306 is shown exiting a distal end of the support 304. In FIG. 3C, the cylindrical base 302 is just coming into contact with the distal end 310 of the ophthalmologic instrument.

In FIG. 3D, the cylindrical base 302 is engaged with the distal end 310 of the ophthalmologic instrument. A distal end 301 of the small diameter instrument 306 is shown protruding from the support 304. Although an unsupported length 303 of the small diameter instrument 306 extends beyond the support 304, the unsupported length 303 is shorter that it would be without the support 304.

Because of the resilient material, the cylindrical base 302 deforms slightly, and conforms to an outer surface of the distal end 310 of the ophthalmologic instrument. The resilient material thereby provides a retaining force that holds the support device 300 on the distal end 310 of the ophthalmologic instrument. Similar to examples discussed above, a level of resilience is selected by choosing an appropriate resilient material. One of ordinary skill in the art, having the benefit of the present disclosure, will recognize that a resilience high enough to provide a desired stiffness to the support 304 and hold the small diameter instrument 306 to at least a level as stiff or stiffer than an equivalent 20 gauge instrument is desired. At the same time, a level of resilience is selected for the cylindrical base 302 to permit deformation and engagement with the distal end 310 of the ophthalmologic instrument.

Although the invention is not so limited, a cone shaped profile of the distal end 310 of the ophthalmologic instrument is shown. In one example, the cylindrical base 302 includes one or more interior profile elements configured to substantially match an exterior profile of the distal end 310. In the example of FIGS. 3A-3D, the one or more interior profile elements includes a single continuous contoured interior surface of the cylindrical base 302 that is structured to conform to substantially match the distal end 310. Other examples may include one or more interior profile elements similar to elements 220 described in FIG. 2A-2D. An advantage of a single continuous contoured interior surface includes case of manufacture. An advantage of one or more interior profile elements similar to elements 220 includes providing focused points of contact that improve stability, over a surface that may include imperfections of texture.

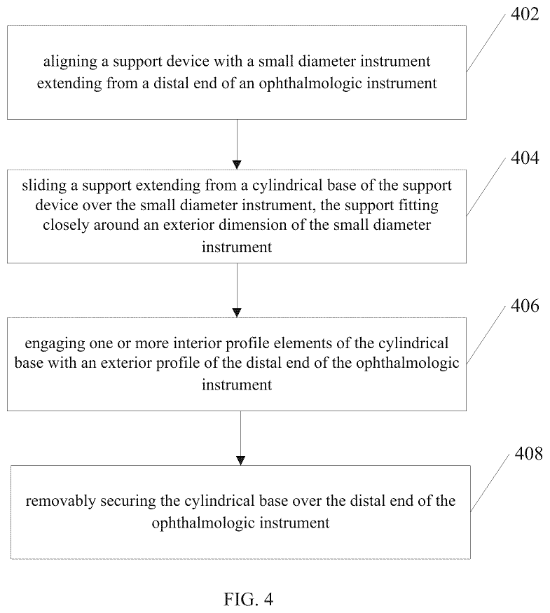

FIG. 4 shows a flow diagram of one example method of supporting a small diameter instrument extending from a distal end of an ophthalmologic instrument. In operation 402, a support device is aligned with a small diameter instrument extending from a distal end of an ophthalmologic instrument. In operation 404, a support extending from a cylindrical base of the support device is slid over the small diameter instrument, the support fitting closely around an exterior dimension of the small diameter instrument. In operation 406, one or more interior profile elements of the cylindrical base are engaged with an exterior profile of the distal end of the ophthalmologic instrument, and in operation 408, the cylindrical base is removably secured over the distal end of the ophthalmologic instrument.

Removable securing provides an advantage over support devices that are not removable. Support devices as described can be removed from a first ophthalmologic instrument and installed on a second ophthalmologic instrument. This reduces cost by eliminating a need for a separate support device for each ophthalmologic instrument. It also provides an ability to use support where needed, and to remove support for procedures where extra support is not needed. A removable support device can be manufactured to fit any new handle configuration of an ophthalmologic instrument, which is easier than re-designing an entire ophthalmologic instrument. Multiple support devices can be included with different lengths of support extending from a cylindrical base. A desired level of support may then be selected by choosing one of a set of different support length options that may be included with a single ophthalmologic instrument.

To better illustrate the method and apparatuses disclosed herein, a non-limiting list of embodiments is provided here:

Example 1 includes a support device for an ophthalmologic instrument. The support device includes a cylindrical base. The cylindrical base includes one or more interior profile elements configured to substantially match an exterior profile of a distal end of an ophthalmologic instrument. The support device includes a support extending from the cylindrical base, the support having an interior dimension that is configured to fit closely around an exterior dimension of a small diameter instrument extending from the distal end of the ophthalmologic instrument.

Example 2 includes the support device of Example 1, wherein the one or more interior profile elements include four interior profile elements.

Example 3 includes the support device of any one of examples 1-2, wherein the four interior profile elements are angularly spaced apart at 90 degrees from one another.

Example 4 includes the support device of any one of examples 1-3, wherein the interior profile elements are configured to substantially match an exterior profile of a cone shaped distal end of the ophthalmologic instrument.

Example 5 includes the support device of any one of examples 1-4, wherein the support is configured to fit closely around a 25 gauge instrument extending from the distal end of the ophthalmologic instrument.

Example 6 is a support device for an ophthalmologic instrument. The support device includes a cylindrical base. The cylindrical base includes one or more interior profile elements configured to substantially match an exterior profile of a distal end of an ophthalmologic instrument and a latching mechanism to hold the cylindrical base to the distal end of the ophthalmologic instrument. The support device also includes a support extending from the cylindrical base, the support having an interior dimension that is configured to fit closely around an exterior dimension of a small diameter instrument extending from the distal end of the ophthalmologic instrument.

Example 7 includes the support device of Example 6, wherein the latching mechanism includes a protrusion and a corresponding socket.

Example 8 includes the support device of any one of examples 6-7, wherein the protrusion is on the distal end of an ophthalmologic instrument, and the corresponding socket is on the cylindrical base.

Example 9 includes the support device of any one of examples 6-8, wherein the latching mechanism includes a resilient material as a portion of the cylindrical base, and an engaging feature on the distal end of the ophthalmologic instrument.

Example 10 includes the support device of any one of examples 6-9, wherein the one or more interior profile elements include four interior profile elements.

Example 11 includes the support device of any one of examples 6-10, wherein the one or more interior profile elements are angularly spaced at equal angles apart.

Example 12 includes the support device of any one of examples 6-11, wherein the support is configured to fit closely around a 25 gauge instrument extending from the distal end of the ophthalmologic instrument.

Example 13 is a support device for an ophthalmologic instrument. The support device includes a cylindrical base. The cylindrical base includes one or more interior profile elements configured to substantially match an exterior profile of a distal end of an ophthalmologic instrument and an interior surface formed from a resilient material, wherein the interior surface includes an interior dimension that is configured to provide an interference fit with the distal end of the ophthalmologic instrument. The support device also includes a support extending from the cylindrical base, the support having an interior dimension that is configured to fit closely around an exterior dimension of a small diameter instrument extending from the distal end of the ophthalmologic instrument.

Example 14 includes the support device of Example 13, wherein the cylindrical base is integrally molded from the resilient material.

Example 15 includes the support device of any one of examples 13-14, wherein the one or more interior profile elements include one continuous contoured interior surface of the cylindrical base.

Example 16 includes the support device of any one of examples 13-15, wherein the one or more interior profile elements include four interior profile elements.

Example 17 includes a method of supporting a small diameter instrument extending from a distal end of an ophthalmologic instrument. The method includes aligning a support device with a small diameter instrument extending from a distal end of an ophthalmologic instrument, sliding a support extending from a cylindrical base of the support device over the small diameter instrument, the support fitting closely around an exterior dimension of the small diameter instrument, engaging one or more interior profile elements of the cylindrical base with an exterior profile of the distal end of the ophthalmologic instrument, and removably securing the cylindrical base over the distal end of the ophthalmologic instrument.

Example 18 includes the method of Example 17, wherein sliding the support extending from the cylindrical base of the support device over the small diameter instrument includes sliding a support over a 25 gauge small diameter instrument.

Example 19 includes the method of any one of Examples 17-18, wherein removably securing the cylindrical base over the distal end of the ophthalmologic instrument includes mating a protrusion on the distal end of the ophthalmological instrument with a recess on the cylindrical base.

Example 20 includes the method of any one of Examples 17-18, wherein removably securing the cylindrical base over the distal end of the ophthalmologic instrument includes deforming a resilient surface of the cylindrical base over a rigid surface of the distal end of the ophthalmological instrument.

Example 21 includes the method of any one of Examples 17-20, wherein the ophthalmologic instrument is a first ophthalmologic instrument, and further including removing the cylindrical base and the support device from the first ophthalmologic instrument; and aligning the support device with a second small diameter instrument extending from a second distal end of a second ophthalmologic instrument, sliding the support extending from the cylindrical base of the support device over the second small diameter instrument, the support fitting closely around an exterior dimension of the second small diameter instrument, engaging one or more interior profile elements of the cylindrical base with a second exterior profile of the second distal end of the second ophthalmologic instrument, and removably securing the cylindrical base over the second distal end of the second ophthalmologic instrument.

These and other examples and features of the present infusion devices, and related methods will be set forth in part in the above detailed description. This overview is intended to provide non-limiting examples of the present subject matter—it is not intended to provide an exclusive or exhaustive explanation.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A support device for an ophthalmologic instrument, comprising:
a cylindrical base, including;
one or more interior profile elements configured to substantially match an exterior profile of a distal end of an ophthalmologic instrument;
a support extending from the cylindrical base, the support having an interior dimension that is configured to fit closely around an exterior dimension of a small diameter instrument extending from the distal end of the ophthalmologic instrument.

2. The support device of claim 1, wherein the one or more interior profile elements include four interior profile elements.

3. The support device of claim 2, wherein the four interior profile elements are angularly spaced apart at 90 degrees from one another.

4. The support device of claim 1, wherein the interior profile elements are configured to substantially match an exterior profile of a cone shaped distal end of the ophthalmologic instrument.

5. The support device of claim 1, wherein the support is configured to fit closely around a 25 gauge instrument extending from the distal end of the ophthalmologic instrument.

6. A support device for an ophthalmologic instrument, comprising:
a cylindrical base, including;
one or more interior profile elements configured to substantially match an exterior profile of a distal end of an ophthalmologic instrument;
a latching mechanism to hold the cylindrical base to the distal end of the ophthalmologic instrument;
a support extending from the cylindrical base, the support having an interior dimension that is configured to fit closely around an exterior dimension of a small diameter instrument extending from the distal end of the ophthalmologic instrument.

7. The support device of claim 6, wherein the latching mechanism includes a protrusion and a corresponding socket.

8. The support device of claim 7, wherein the protrusion is on the distal end of an ophthalmologic instrument, and the corresponding socket is on the cylindrical base.

9. The support device of claim 6, wherein the latching mechanism includes a resilient material as a portion of the cylindrical base, and an engaging feature on the distal end of the ophthalmologic instrument.

10. The support device of claim 6, wherein the one or more interior profile elements include four interior profile elements.

11. The support device of claim 10, wherein the one or more interior profile elements are angularly spaced at equal angles apart.

12. The support device of claim 6, wherein the support is configured to fit closely around a 25 gauge instrument extending from the distal end of the ophthalmologic instrument.

13. A support device for an ophthalmologic instrument, comprising:
a cylindrical base, including;
one or more interior profile elements configured to substantially match an exterior profile of a distal end of an ophthalmologic instrument;
an interior surface formed from a resilient material, wherein the interior surface includes an interior dimension that is configured to provide an interference fit with the distal end of the ophthalmologic instrument;
a support extending from the cylindrical base, the support having an interior dimension that is configured to fit closely around an exterior dimension of a small diameter instrument extending from the distal end of the ophthalmologic instrument.

14. The support device of claim 13, wherein the cylindrical base is integrally molded from the resilient material.

15. The support device of claim 14, wherein the one or more interior profile elements include one continuous contoured interior surface of the cylindrical base.

16. The support device of claim 13, wherein the one or more interior profile elements include four interior profile elements.

17. A method of supporting a small diameter instrument extending from a distal end of an ophthalmologic instrument, comprising:
aligning a support device with a small diameter instrument extending from a distal end of an ophthalmologic instrument;
sliding a support extending from a cylindrical base of the support device over the small diameter instrument, the support fitting closely around an exterior dimension of the small diameter instrument;
engaging one or more interior profile elements of the cylindrical base with an exterior profile of the distal end of the ophthalmologic instrument; and
removably securing the cylindrical base over the distal end of the ophthalmologic instrument.

18. The method of claim 17, wherein sliding the support extending from the cylindrical base of the support device over the small diameter instrument includes sliding a support over a 25 gauge small diameter instrument.

19. The method of claim 17, wherein removably securing the cylindrical base over the distal end of the ophthalmologic instrument includes mating a protrusion on the distal end of the ophthalmological instrument with a recess on the cylindrical base.

20. The method of claim 17, wherein removably securing the cylindrical base over the distal end of the ophthalmologic instrument includes deforming a resilient surface of the cylindrical base over a rigid surface of the distal end of the ophthalmological instrument.

21. The method of claim 17, wherein the ophthalmologic instrument is a first ophthalmologic instrument, and further including removing the cylindrical base and the support device from the first ophthalmologic instrument; and
aligning the support device with a second small diameter instrument extending from a second distal end of a second ophthalmologic instrument;
sliding the support extending from the cylindrical base of the support device over the second small diameter instrument, the support fitting closely around an exterior dimension of the second small diameter instrument;
engaging one or more interior profile elements of the cylindrical base with a second exterior profile of the second distal end of the second ophthalmologic instrument; and
removably securing the cylindrical base over the second distal end of the second ophthalmologic instrument.
